(19) United States
(12) Patent Application Publication
(10) Pub. No.: US 2025/0177659 A1
(43) Pub. Date: Jun. 5, 2025

(54) A DELIVERY SITE DETECTOR AND A MEDICAMENT DELIVERY DEVICE THAT COMPRISES THE DELIVERY SITE DETECTOR

(71) Applicant: SHL Medical AG, Zug (CH)

(72) Inventors: Yu-Lin Song, New Taipei City (TW); Shih-Hsun Tu, Taoyuan City (TW); Pei-Chi Hu, Taoyuan City (TW); Chun-Yi Lee, Taoyuan City (TW); Alexander Stiegler, Taoyuan City (TW)

(21) Appl. No.: 18/841,514

(22) PCT Filed: Feb. 20, 2023
(86) PCT No.: PCT/EP2023/054238

§ 371 Date: Aug. 26, 2024

Related U.S. Application Data
(60) Provisional application No. 63/316,021, filed on Mar. 3, 2022.

(30) Foreign Application Priority Data
Aug. 19, 2022 (EP) 22191127.4

(51) Int. Cl. A61M 5/42; A61M 5/46

(52) U.S. Cl. CPC A61M 5/427; A61M 5/46; A61M 2205/0233; A61M 2205/0238; A61M 2205/0294; A61M 2205/3375

(57) ABSTRACT

The present disclosure provides a delivery site detector configured to be attached to a medicament delivery device. The delivery site detector includes an ultrasonic transmitter and an ultrasonic receiver. The ultrasonic transmitter is configured to transmit an ultrasonic wave to a delivery site. The ultrasonic receiver is configured to receive an ultrasonic wave reflected by the delivery site. The ultrasonic transmitter comprises a frequency generator configured to generate and transmit ultrasonic waves with at least two different ultrasound frequencies.

### CROSS-REFERENCE TO RELATED APPLICATIONS

The present application is a U.S. National Phase Application pursuant to 35 U.S.C. § 371 of International Application No. PCT/EP2023/054238 filed Feb. 20, 2023, which claims priority to (i) U.S. Provisional Patent Application No. 63/316,021 filed Mar. 3, 2022, and (ii) European Patent Application No. 22161127.4 filed Aug. 19, 2022. The entire disclosure contents of these applications are herewith incorporated by reference into the present application.

### TECHNICAL FIELD

The present disclosure relates to delivery site detectors that are configured to be attached to a medicament delivery device, and particularly to delivery site detectors configured to assist a user of the device in selecting an appropriate site for medicament delivery.

### BACKGROUND

Medicament delivery devices, especially those designed for medicament delivery by self-administration, have been on the market for a number of years. In order for the devices to be handled by non-professionals, they have to be easy to use and intuitive. Further, since many of the drugs are vital or at least very important to the patient there is a desire from physicians and other professionals to ensure that health care providers or the patients correctly perform medicament delivery. In particular, choosing the correct delivery site to administer a dose of medicament, especially where skin invasive delivery is to occur, can be medicament dependent. In other words, some medicaments are only effective when delivered to the correct tissue, for example, subcutaneous vs. intermuscular vs. intravenous.

Likewise, repetitive drug delivery to the same delivery site could lead to nodule and lump formation, which could then change or influence the drug absorption rate in the particular tissue. This could then cause an unknown and uncontrollable dose of the drug to be absorbed. Or an inadvertent administration of a particular medicament to a vein or blood vessel could lead to undesirable consequences. Such safety considerations concerning choosing an injection site cannot be overstated. For example, if a diabetic were to inject insulin into a muscle layer, this could lead to hypoglycaemia. Or, if epinephrine is delivered into the subcutaneous layer, this could possibly lead to a life-threatening situation.

It is therefore desirable to assist the user of a medicament delivery device, especially a non-medically trained user, in determining the correct drug delivery site when a skin invasive drug delivery is to occur. The art recognizes a number of proposed technologies for choosing a correct injection site, including the use of an ultrasonic scanner. For example, U.S. Pat. 7,722,535 discloses a single frequency scanner for assisting a user of a medicament delivery device in choosing a site for a skin invasive action in such a way that an appropriate area of the skin is chosen for the action before the skin invasive action is performed. However, these early devices do not provide the accuracy now needed for the wide variety of injectable drugs on the market. Likewise, there is a strong desire to use supplemental devices attachable to existing medicament delivery devices to determine the optimal delivery site. In this way, design modification of approved and existing devices is not necessary, thus avoiding potential government regulatory issues.

### SUMMARY

The invention is defined by the appended claims, to which reference should now be made.

In the present disclosure, when the term “distal direction” is used, this refers to the direction pointing away from the dose delivery site during use of the injection device. When the term “distal part/end” is used, this refers to the part/end of the delivery device, or the parts/ends of the members thereof, which during use of the medicament delivery device is/are located furthest away from the dose delivery site. Correspondingly, when the term “proximal direction” is used, this refers to the direction pointing towards the dose delivery site during use of the injection device. When the term “proximal part/end” is used, this refers to the part/end of the delivery device, or the parts/ends of the members thereof, which during use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component. Similarly, “radial” or “radially” refer to a direction extending radially relative to the axis, and “rotation”, “rotational” and “rotationally” refer to rotation relative to the axis.

There is hence provided a delivery site detector configured to be attached to a medicament delivery device. The delivery site detector comprises an ultrasonic transmitter and an ultrasonic receiver. The ultrasonic transmitter is configured to transmit an ultrasonic wave to a delivery site. The ultrasonic receiver is configured to receive an ultrasonic wave reflected by the delivery site.

The ultrasonic transmitter comprises a frequency generator configured to generate and transmit ultrasonic waves with at least two different ultrasound frequencies.

Preferably, according to another embodiment, the frequency generator comprises a plurality of stacked dielectric or piezoelectric layers.

Preferably, according to another embodiment, at least two of the plurality of stacked dielectric or piezoelectric layers have different thicknesses.

Alternatively, according to another embodiment, all layers of the plurality of stacked dielectric or piezoelectric layers have the same thickness.

Preferably, according to another embodiment, each of the plurality of stacked dielectric or piezoelectric layers is bonded to its adjacent dielectric or piezoelectric layers with conductive material coating on each of the plurality of stacked dielectric or piezoelectric layers.

Preferably, according to another embodiment, an indicator operably connected to the delivery site detector; a processor connected to the indicator and the delivery site detector; a battery connected to the delivery site detector, the indicator, and the processor. The processor is configured to control the indicator to provide at least a visual, audible, and/or tactile indication, and/or to send out a signal containing data based on a detection of the delivery site detector.

Another aspect of the invention provides a delivery member guard of a medicament delivery device that comprises the delivery site detector.

Preferably, according to another embodiment, the delivery member guard comprises a sleeve. The sleeve comprises a proximal wall configured to contact the delivery site. An aperture is arranged in the proximal wall of the sleeve and is configured to be passed through by a medicament delivery member of the medicament delivery device during use of the medicament delivery device. The ultrasonic transmitter and the ultrasonic receiver are attached to the proximal wall of the sleeve.

Another aspect of the invention provides a housing of a medicament delivery device, wherein the housing is configured to accommodate a medicament container of the medicament delivery device, the housing comprising the delivery site detector.

Another aspect of the invention provides medicament delivery device comprising either the delivery member guard as mentioned above, or the housing as mentioned above.

Preferably, according to another embodiment, the medicament delivery device comprising: an indicator operably connected to the delivery site detector; a processor connected to the indicator and the delivery site detector; a battery connected to the delivery site detector, the indicator, and the processor. The processor is configured to control the indicator to provide at least a visual, audible, and/or tactile indication based on a detection of the delivery site detector, and/or to send out a signal containing data based on a detection of the delivery site detector.

In this embodiment, the delivery site detector does not need to be arranged with at least one electronics that the medicament delivery device has. For example, as the medicament delivery device in this embodiment has the indicator, the delivery site detector does not need to comprise the indicator as mentioned above. In a preferred example, the delivery site detector in this embodiment does not have the indicator, the battery, and the processor.

Preferably, according to another embodiment, the medicament delivery device comprising a disposable cassette unit and a reusable drive unit releasably attached to the cassette unit. The delivery site detector is arranged in the cassette unit. The battery, the indicator and the processor are arranged in the drive unit.

Preferably, according to another embodiment, the medicament delivery device comprising a removable protective cap for sealing a medicament delivery member. The protective cap comprises a portion of a switch that when activated provides power to the delivery site detector.

Preferably, according to another embodiment, the switch comprises an insulating strip having a fixed end attached to the protective cap and a free end positioned between two electrical contacts within a circuit that is operatively connected to the battery and the detector.

Preferably, according to another embodiment, the medicament delivery device comprising a depth adjuster configured to determine the furthest distance that a medicament delivery member can reach.

Preferably, according to another embodiment, the depth adjuster is automatically controlled and adjusted based on an adjustment signal received from the detector.

Preferably, according to another embodiment, when the medicament delivery device comprises the processor, the depth adjuster connects to the processor. The depth adjuster operably connects to either a medicament container or a delivery member guard. The processor is configured to control the depth adjuster to move the medicament container when the depth adjuster operably connects to the medicament container to a predetermined position based on the detection of the delivery site detector; or the processor is configured to control the depth adjuster to move the delivery member guard, when the depth adjuster operably connects to the delivery member guard, to a predetermined position based on the detection of the delivery site detector.

Another aspect of the invention provides a method of identifying a medicament delivery site. The method comprising the following steps in the following order: providing a medicament delivery device with the delivery site detector; transmitting ultrasonic waves from the delivery site detector with at least two different ultrasonic frequencies towards a selected delivery site; receiving the ultrasonic waves from the selected delivery site; determining, based on the received ultrasonic waves, whether the selected delivery site is suitable for delivery of the contained medicament of the medicament delivery device.

Preferably, according to another embodiment, the method further comprising a step of: determining whether the penetration depth of a delivery member of the medicament delivery device needs to be adjusted if the selected delivery site is suitable for delivery of the contained medicament of the medicament delivery device.

Preferably, according to another embodiment, the method further comprising a step of: providing at least one audible, visual, and/or tactile indication, and/or sending out a signal containing data based on the determination of whether the selected delivery site is suitable for delivery of the contained medicament of the medicament delivery device.

Preferably, according to another embodiment, the step of transmitting ultrasonic waves from the delivery site detector further comprises the following steps when the delivery site detector comprises the plurality of stacked dielectric or piezoelectric layers: selectively providing a voltage across varying numbers of dielectric or piezoelectric layers to generate ultrasonic waves with at least two different ultrasonic frequencies.

Preferably, according to another embodiment, the step of transmitting ultrasonic waves from the delivery site detector further comprises the following steps when at least two layers of the plurality of stacked dielectric or piezoelectric layers have different thicknesses: selectively providing a voltage across piezoelectric layers of different thicknesses to generate ultrasonic waves with at least two different ultrasonic frequencies.

Another aspect of the invention provides a medicament delivery device that has a delivery mechanism positioned within a housing of the medicament delivery device and includes an outlet orifice in fluid communication with a container of medicament located within the housing and which is operatively connected to the delivery mechanism.

Preferably, according to another embodiment, an indicator is operatively connected to the housing as an integral part of the detector or as a separate signalling device that can be used to provide feedback to a user regarding the suitability of a chosen medicament delivery site.

Preferably, according to another embodiment, the indicator has a first state, a second state, and a third state. The first state shows that the detector is not activated, the second state indicates that the delivery site is suitable, and the third state signifies that the delivery site is not suitable. After completion of the medicament delivery, the status indicator can return to the first state.

Preferably, according to another embodiment, the delivery site detector has a transmitter and a receiver operatively associated with the status indicator, where the detector uses ultrasonic wave measurement to control the status indicator and, as such, the user's placement of the medicament delivery device at a correct delivery site.

Preferably, according to another embodiment, the medicament delivery device can also have a delivery member guard configured to extend proximally from a proximal end of the housing such that the delivery member guard covers the medicament outlet orifice. This delivery member guard can be locked from axial movement except when the status indicator is in the second state. After completion of the medicament delivery, the delivery member guard can again be locked in a proximally extending position to cover the outlet orifice and to prevent further medicament delivery.

Preferably, according to another embodiment, the detector can be configured to transmit variable frequencies of sound waves that will reflect the presence of bone, veins, intradermal tissue, muscle, and subcutaneous tissue and can determine a change in distance between the outlet orifice and the injection site during medicament delivery, where the change in distance will trigger an alarm signal if the chosen delivery site is not correct or suitable.

The detector of the present disclosure can be used in medicament delivery devices that perform a variety of skin invasive actions, such as a delivery device that causes a physical object, e.g. a needle, a fluid, a drug and/or any other suitable kind of object to cross a boundary defined by an outer surface of the skin of the mammal. Whether or not the particular location chosen for such an invasive action is appropriate or suitable may depend on a number of factors, including, but not limited to, the kind of skin invasive action, properties of an outer surface of the skin, the ability of one or more subcutaneous layers to absorb a specific drug, distance to blood vessels, whether or not the site has been used recently for a similar skin invasive action, etc.

Preferably, according to another embodiment, the medicament delivery device is an auto-injector, manual pen injector, or an infusion device.

Preferably, according to another embodiment, the auto-injector can be a pen type auto-injector, an on-body device or an injection pump.

Preferably, according to another embodiment, the medicament container is a syringe or a cartridge.

Another aspect of the invention provides an ultrasonic detector comprising an ultrasonic transmitter and an ultrasonic receiver; the ultrasonic transmitter is configured to transmit an ultrasonic wave to a target site; the ultrasonic receiver is configured to receive an ultrasonic wave reflected by the target site; the frequency generator is configured to generate and transmit ultrasonic waves with at least two different ultrasound frequencies; the frequency generator comprises a plurality of stacked dielectric or piezoelectric layers; and wherein each of the plurality of stacked dielectric or piezoelectric layers is bonded to its adjacent dielectric or piezoelectric layer(s) with conductive material.

Preferably, according to another embodiment, the plurality of stacked dielectric or piezoelectric layers have the same thickness.

Alternatively, according to another embodiment, the plurality of stacked dielectric or piezoelectric layers have different thicknesses.

Preferably, according to another embodiment, the frequency generator is configured to generate and transmit ultrasonic waves with at least two different ultrasound frequencies by selectively providing an electrical voltage across varying numbers of the stacked dielectric or piezoelectric layers.

Preferably, according to another embodiment, the frequency generator is configured to generate and transmit ultrasonic waves with at least two different ultrasound frequencies by selectively providing an electrical voltage across varying layers of the stacked dielectric or piezoelectric layers.

Preferably, according to another embodiment, the conductive material is coated on each of the plurality of stacked dielectric or piezoelectric layers.

Preferably, according to another embodiment, the ultrasonic detector comprising: an indicator operably connected to the ultrasonic detector; a processor connected to the indicator and the ultrasonic detector; a battery connected to the ultrasonic detector, the indicator, and the processor; the processor is configured to control the indicator to provide at least a visual, audible and/or tactile indication, and/or to send out a signal containing data based on a detection of the ultrasonic detector.

As the frequency generator is configured to generate and transmit ultrasonic waves with at least two different ultrasound frequencies, the frequency generator can be also used in an ultrasonic cleaning device, an aerosol generator, ultrasonic motor, and ultrasonic extractor.

In a preferred example, the aerosol generator can be a medicament delivery device, e.g., an inhalation device or a medical sprayer, or an electronic cigarette. In the example, the transmit ultrasonic waves are configured to atomize liquid substance, e.g., medicament or tobacco product. The ultrasonic waves with multiple frequencies can generate mist droplets in different sizes, thus, the mist droplets can be distributed broadly.

Furthermore, as the at least two different ultrasound frequencies by selectively providing an electrical voltage across varying layers of the stacked dielectric or piezoelectric layers, the size of the frequency generator can be compact, thus, the frequency generator can be also used in a hand-hold, portable device.

The medicament delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary types of drugs that could be included in the medicament delivery devices described herein include, but are not limited to, antibodies, proteins, fusion proteins, peptibodies, polypeptides, pegylated proteins, protein fragments, protein analogues, protein variants, protein precursors, and/or protein derivatives. Exemplary drugs that could be included in the medicament delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-la (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatitis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)). Pharmaceutical formulations including, but not limited to, any drug described herein are also contemplated for use in the medicament delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

Furthermore, all terms used in the claims are to be interpreted according to their ordinary meaning in the technical field, unless explicitly defined otherwise herein. All references to “a/an/the element, apparatus, component, means, etc.” are to be interpreted openly as referring to at least one instance of the element, apparatus, component, means, etc., unless explicitly stated otherwise.

### BRIEF DESCRIPTION OF THE DRAWINGS

Embodiments of the inventive concept will now be described, by way of example only, with reference to the accompanying drawings, in which:

FIG. 1A illustrates a medicament delivery device of the present disclosure having an integral or built-in detector;

FIG. 1B is a cross-sectional view of the embodiment of FIG. 1A;

FIG. 2A illustrates another embodiment of a medicament delivery device of the present disclosure having an integral or built-in detector, where the transmitter and receiver are located on a delivery member.

FIG. 2B illustrates the embodiment of FIG. 2A in a cross-sectional view.

FIG. 3 illustrates another possible embodiment where the detector is part of a supplemental device attached to an existing design of a medicament delivery device.

FIG. 4 represents how the medicament delivery device and detector of the present disclosure interact with a cross-sectional view of the tissue at a suitable injection site.

FIG. 5 illustrates another embodiment of a medicament delivery device of the present disclosure having an integral or built-in detector, where the transmitter and receiver are located on a delivery member guard. In this example, the medicament delivery device is formed by a cassette unit and a drive unit. The detector is located on the cassette unit.

FIG. 6 shows a flow chart of an operation of a method of identifying a medicament delivery site with the delivery site detector in one embodiment.

FIGS. 7A-7D show an example of a frequency generator of the detector and the charts of its output signal.

### DETAILED DESCRIPTION

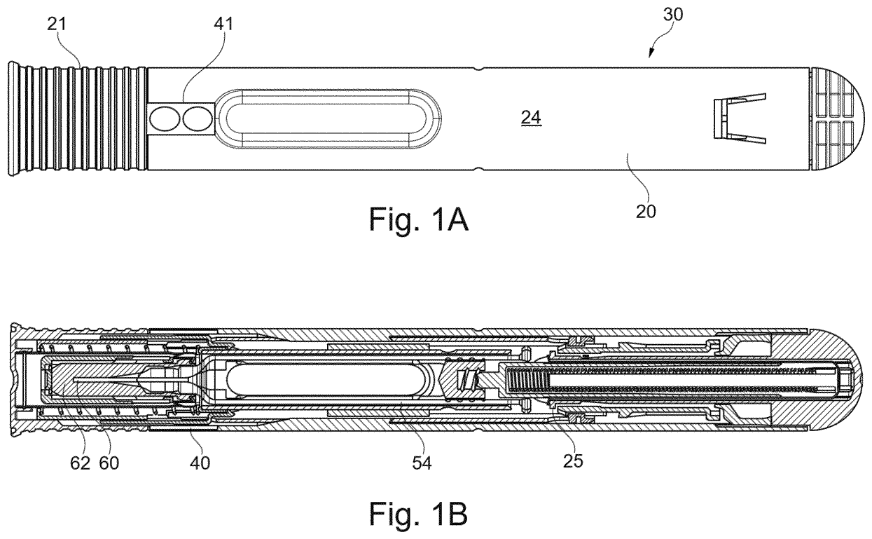

FIGS. 1A-7D illustrate a delivery site detector configured to be attached to a medicament delivery device. The delivery site detector comprises an ultrasonic transmitter and an ultrasonic receiver. The ultrasonic transmitter is configured to transmit an ultrasonic wave to a delivery site, and the ultrasonic receiver is configured to receive an ultrasonic wave reflected by the delivery site. In a preferred example, the ultrasonic transmitter comprises a frequency generator configured to generate and transmit ultrasonic waves with at least two different ultrasonic frequencies.

In one example, the ultrasonic transmitter and the ultrasonic receiver are transducers with dielectric or piezoelectric material. In one example, the ultrasonic transmitter and the ultrasonic receiver are formed by one transducer with dielectric or piezoelectric material. For example, an electrical circuit can connect to a piezoelectric crystal by wire. For performing the function of the ultrasonic transmitter, the circuit provides a voltage to the piezoelectric crystal. The electrical energy is then converted to a mechanical vibration of the piezoelectric crystal, thereby ultrasonic waves are generated. When reflected ultrasonic waves hit the piezoelectric crystal, a mechanical vibration caused by the reflected ultrasonic waves changes the electrical signal in the electrical circuit, the function of the ultrasonic receiver is thus provided. In another example, the ultrasonic transmitter and the ultrasonic receiver can be formed by two independent transducers.

The delivery site detector is configured to determine whether a chosen skin site is suitable for a skin invasive action, e.g., being penetrated by a needle with a certain penetration length or having a specific medicament delivered. In determining the suitability of a delivery or injection site, the detector of the present disclosure uses ultrasonic waves, preferably at two more frequencies, to measure one or more parameters relating to a part of the skin or subcutis of the subject receiving delivery of the medicament. This obtained information is then processed so as to characterize the selected medicament delivery site location. In situations where it is determined that the delivery site for some reason is not suitable, the user of the medicament delivery device will be notified that another site should be chosen.

If and when the user chooses a different site, then the procedure executed by the detector will be repeated accordingly until a suitable site is found. Repeating the procedure may be automatic and may start when the user physically places the medicament delivery device against another chosen delivery site. Alternatively, repeating the procedure may require the user to manually activate the detector in order to evaluate a new site.

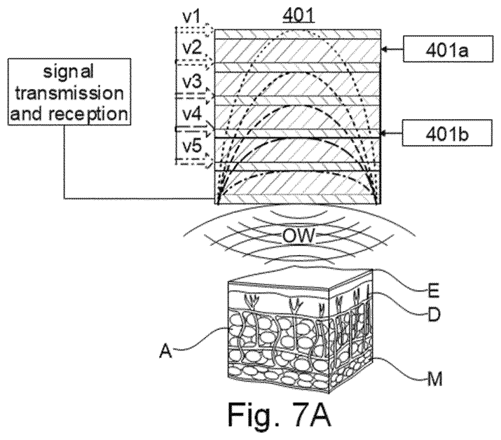

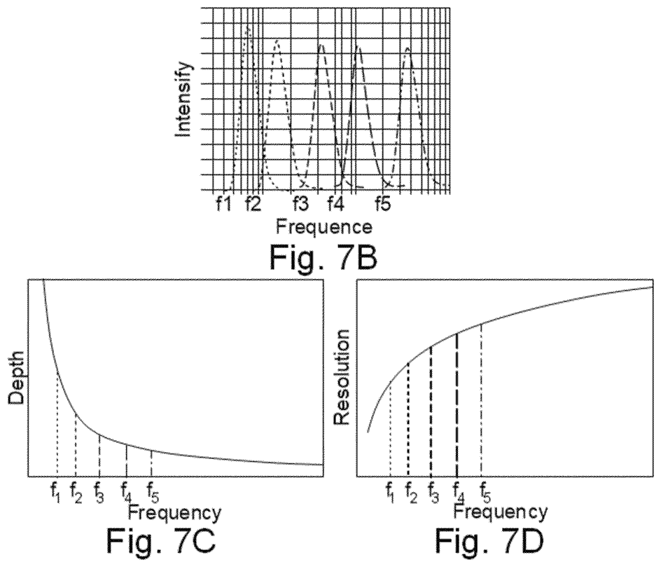

In a preferred example, the ultrasonic transmitter comprises a frequency generator configured to generate and transmit ultrasonic waves with at least two different ultrasonic frequencies. This use of variable ultrasonic frequencies can effectively detect the exact location of veins inside the skin. Indeed, the use of three frequency ranges can be effective, where a low frequency range can be used to detect the deeper organ with lower resolution, medium frequency range can be used to detect the subcutaneous layer with a medium resolution, and higher frequency range can be used to detect the intradermal tissue with higher resolution than the lower or medium frequency ranges, as shown in FIGS. 7B-7C. The preferred frequency range is from about 5 MHz to about 50 MHz. Use of a greater number of frequencies allows more precise detection of different characteristics of the analyzed delivery site. The frequencies used for medical imaging are generally in the range of 1 to 18 MHz. Higher frequencies have a correspondingly smaller wavelength and can be used to make sonograms with more precise details. However, the attenuation of the sound wave is increased at higher frequencies, so in order to have better penetration of deeper tissues, a lower frequency range (3-5 MHz) is commonly used. The detector of the present detector is not limited by one such frequency and can employ multiple frequencies of ultrasound to achieve both high resolution and depth of tissue evaluation.

The characteristics of the skin and/or one or more subcutaneous layers of a user vary between various body parts. Thus, the skin of a stomach area of a person will be distinguishable from the skin of the thighs of the same person, primarily because the position of the subcutaneous fat layer is different for a stomach area and a thigh area. Furthermore, some characteristics of the skin and/or one or more subcutaneous layers may vary across a specific body part. Thus, the skin of an inner part of a thigh may be distinguishable from the skin of an outer part of the same thigh. Furthermore, the skin of a specific person may comprise specific and unique features which are characteristic for a specific site of the skin of that person. Such specific features may, e.g., be birth marks, lumps or slumps in the skin, damaged areas of the skin and/or subcutaneous layers, the positions of one or more inner organs, the thickness of a layer of fat or variations in the thickness of a layer of fat, etc. Therefore, the delivery site detector can detect whether the selected delivery site is matched with the predetermined body part.

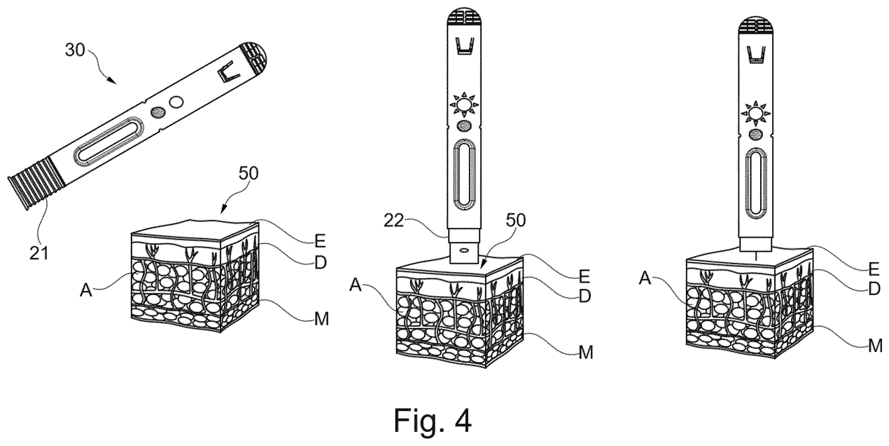

The variable frequency capability of the detector of the present disclosure can be adjusted or predetermined to determine exactly which part of the skin of the user is selected for evaluation as a possible medicament delivery site. Such an identification of the part of the skin may be based on a comparison between the measured and processed parameters on one hand and previously obtained data on the other hand. It is commonly known that the speed of sound varies as it travels through different materials and is dependent on the acoustical impedance of the material. The sound reflects and echoes off parts of the tissue layers, for example the epidermis (E), dermis (D), adipose tissue (A) and muscle (M) (see FIG. 4). The wave echo received by the receiver in the detector may be recorded and displayed as an image or calculation of the tissue layers to the user.

In a preferred example, the frequency generator 401 comprises a plurality of stacked dielectric or piezoelectric layers 401, as shown in FIG. 7A. Preferably, the frequency generator can be a transducer that utilizes piezoelectric crystals which convert electrical energy into mechanical/sound wave energy because such crystals change geometric size/vibrate when an electrical voltage is applied.

In the preferred example, the frequency generator 401 comprises a plurality of stacked piezoelectric layers 401. As the frequency of the output sound wave is dependent on how thick a piezoelectric layer the electrical energy passes through, when the electrical energy passes through the different thicknesses of layers of piezoelectric crystals, sound waves with multiple frequencies can be generated. As shown in FIG. 7A, providing the input voltage selectively across varying numbers of piezoelectric layers 401a or across one of the piezoelectric layers 401a causes the selected piezoelectric layer(s) 401a to vibrate, thereby the output waves OW are generated. For example, one of the electrodes can be fixed to the outer surface of the first or the last layer of the stacked of piezoelectric layers, the other electrodes can selectively connect to another stack of piezoelectric layers v1, v2, v3, v4, v5, as shown in FIG. 7A. In this example, the output waves OW are generated with the frequency f1, f2, f3, f4, f5, as shown in FIG. 7B.

In one example, the plurality of stacked piezoelectric layers 401 can be divided from one piezoelectric crystal. In another example, the plurality of stacked piezoelectric layers 401 can be made of bonding multiple piezoelectric crystals all together.

In one example, all layers of the plurality of stacked dielectric or piezoelectric layers 401 have the same thickness. In this example, selectively providing a voltage across varying numbers of piezoelectric layers 401a can generate ultrasonic waves of at least two different ultrasonic frequencies.

In another example, all layers of the plurality of stacked dielectric or piezoelectric layers 401 have different thicknesses compared to one another. In this example, the ultrasonic waves with at least two different ultrasonic frequencies can be generated by either providing voltage across varying numbers of piezoelectric layers as mentioned above, or by selectively vibrating piezoelectric layers of different thicknesses. In this example, frequencies that can be generated will not be limited to be whole-number multiples based on the thickness of a basic frequency, which can allow a greater number of frequencies to be generated. Alternatively, some layers of the plurality of stacked dielectric or piezoelectric layers have the same thickness, and the rest of the plurality of stacked dielectric or piezoelectric layers have different thicknesses compared to one another.

Each of the plurality of stacked dielectric or piezoelectric layers 401a is bonded to its adjacent dielectric or piezoelectric layer(s) with a conductive material 401b, e.g., iron glue, sliver glue, thereby the entire plurality of stacked dielectric or piezoelectric layers 401a is bonded as a whole. In a preferred example, the conductive material 401b is coated on each of the plurality of stacked dielectric or piezoelectric layers 401a, so that each of the plurality of stacked dielectric or piezoelectric layers 401a can bond to its adjacent dielectric or piezoelectric layer(s).

Preferably, each of the plurality of stacked dielectric or piezoelectric layers 401 is bonded to its adjacent dielectric or piezoelectric layer(s) with silver glue 401b. The plurality of stacked dielectric or piezoelectric layers 401 will generate heat during vibration and/or when a current pass through the piezoelectric layers. Therefore, in a preferred example, the conductive material for bonding the plurality of stacked dielectric or piezoelectric layers 401 is a material that can keep its structural and conductive properties within a certain temperature range. For example, most piezoelectric materials are only piezoelectric under about 150 degrees Celsius, and sliver glue remains conductive and adhesive up to about 200 degrees Celsius. Bonding the entire plurality of stacked dielectric or piezoelectric layers 401a with silver glue 401b can thus enable a wide range of use of the frequency generator with the plurality of stacked dielectric or piezoelectric layers 401. Alternatively, if the temperature for using the frequency generator with the plurality of stacked dielectric or piezoelectric layers 401 is predetermined to be a relatively lower temperature, conductive material with a low temperature tolerance can be used to bond the entire plurality of stacked dielectric or piezoelectric layers. For example, iron glue can tolerate about 100 degrees Celsius.

In one example where the frequency generator is a transducer of the plurality of stacked dielectric or piezoelectric layers 401, the detector can be compact and suitable for being used in medicament delivery devices of various sizes, in particular, suitable for being used in portable, self-administration medicament delivery devices. With portable, self-administration medicament delivery devices, e.g., autoinjectors or insulin pumps, which are usually designed to inject into muscle (intramuscular medicament delivery) or skin layer (subcutaneous medicament delivery), it might be not necessary to have a delivery site detector that can detect the position of embryos or bones. As the low frequency range is generated with a relatively thicker piezoelectric material (as shown in FIGS. 7A-7B, if the entire plurality of stacked dielectric or piezoelectric layers 401 vibrates, the lowest frequency of this transducer can be generated) and the low frequency range is used to detect deeper organs, therefore, if the deepest organ for portable, self-administration medicament delivery devices is muscle, the entire thickness of the plurality of stacked dielectric or piezoelectric layers 401 (measured in the direction that each of the plurality of dielectric or piezoelectric layers is stacked, namely, the direction perpendicular to the layer surfaces) can be limited. For example, for detecting a proper delivery site for most portable, self-administration medicament delivery devices, the preferred frequency range will be 5 MHz to 50 MHz. For generating and transmitting the ultrasonic waves within this range, the entire thickness of the plurality of stacked dielectric or piezoelectric layers 401 would be around 20 micrometers to 300 micrometers. Therefore, the detector can be easily attached to a portable, self-administration medicament delivery device that is also usually compact and light without significantly increasing the size of the medicament delivery device and/or having an impact on the medicament delivery operation.

On the other hand, the frequency generator can comprise multiple transducers with a different size, for example, multiple piezoelectric crystals with different thicknesses. In this example, instead of stacking on one another, the multiple transducers can be arranged as an array. In this example, each transducer can be configured to generate a sound wave with one frequency that is different from others which are generated from other transducers when the electrical energy passes through. In this example, ultrasonic waves with multiple frequencies can be generated simultaneously, instead of being generated sequentially using one transducer with the plurality of stacked dielectric or piezoelectric layers as mentioned above.

The reflected ultrasonic waves received by the receiver can be used by a processor to provide images and/or calculations with a two-dimensional cross-section of the tissue being imaged. The ultrasonic wave transmitted by the ultrasonic transmitter encounters the multiple layers of the epidermis (E), dermis (D), adipose tissue (A), and muscle (M) with different densities (acoustical impedance) (see FIG. 4). Accordingly, part of the ultrasonic wave is reflected, and received by the receiver, which preferably is located adjacent to the transmitter. The time t1 it takes for the reflected sound wave (echo) to travel back to the receiver is measured and used to calculate the depth of the tissue interface or structure within the tissue (e.g., a vein) causing the echo. The greater the difference between acoustic impedances, the larger the echo is. As the detector can generate ultrasonic waves with multiple frequencies, tissues can be fully scanned and/or the ambient area of tissue can be detected. For example, if the generated ultrasonic waves are in a single frequency, the detection can only tell that there is no muscle tissue. On the other hand, if the ultrasonic waves with multiple frequencies are generated, as the ultrasonic waves now can reach multiple different positions with different depths under the skin, the detection can for example show that there is a muscle tissue under the fat layer, if the needle can be inserted 2 mm further into this injection site further than the default penetration depth, it can reach the muscle tissue; or clearly show there is a vessel over the muscle tissue. Therefore, the detection from the detector can either indicate or control an adjustment of the penetration depth (will be explained in detail later) or indicate the user to move to another injection site to avoid any damage to the vessel.

In one example, the delivery site detector further comprises an indicator operably connected to the delivery site detector; a processor connected to the indicator and the delivery site detector; and a battery connected to the delivery site detector, the indicator, and the processor. The processor is configured to control the indicator to provide at least a visual, audible and/or tactile indication, and/or to send out a signal containing data based on a detection of the delivery site detector. Alternatively, the delivery site detector can be attached to a medicament delivery device that provides at least one of the processor, the indicator and the battery.

In a preferred example, the received ultrasonic waves are processed by the processor. In this example, the processor is operatively connected to the ultrasonic receiver which receives signals corresponding to reflected sound waves resulting from the transmitted two or more ultrasonic frequencies. For example, the processor uses an algorithm to determine whether or not the chosen delivery site is suitable for the skin invasive action based on the processed parameters. In some cases, the detector can suggest the location of another possible delivery site, e.g., the thigh as opposed to the stomach. As the determination can be based on image recognition technology, the algorithm may be based on machine learning technology.

Furthermore, the processor can also operatively connect to the ultrasonic transmitter. The detector can be programmed to sequentially provide a voltage across varying numbers of the plurality of stacked dielectric or piezoelectric layers 401, or sequentially provide a voltage across different layers of the plurality of stacked dielectric or piezoelectric layers 401 when the stacked dielectric or piezoelectric layers are of varying thicknesses. Alternatively, the detector can provide, based on the previous received ultrasonic waves, a voltage across varying numbers of the plurality of stacked dielectric or piezoelectric layers 401, or across different layers of the plurality of stacked dielectric or piezoelectric layers 401 when the stacked dielectric or piezoelectric layers are of varying thicknesses.

The processor can be configured to execute one or more algorithms that will evaluate the received reflected ultrasonic waves and will determine if the medicament delivery device is positioned at a suitable delivery site. The evaluation and determination by the algorithm are then communicated to the user through a signal sent to the indicator representative of the correctness of the positioning of the medicament delivery device. The indicator then provides a visual, audible, and/or tactile signal to the user when the detector is activated; and/or when the indicator comprises a communication unit, the indicator can send out an information signal to an external device. The processor component in the detector can also be adapted to detect a predetermined event pattern, such as an incorrect delivery site, and to provide notification to an external device that the event has occurred. The control component can also create a time log representing the detected event as a function of time. As such, it is desirable that have a clock function.

The detector of the present disclosure can be used in medicament delivery devices that perform a variety of skin invasive actions, such as a delivery device that causes a physical object, e.g., a needle, a fluid, a drug and/or any other suitable kind of object to cross a boundary defined by an outer surface of the skin of the mammal. Whether or not the particular location chosen for such an invasive action is appropriate or suitable may depend on a number of factors, including, but not limited to, the kind of skin invasive action, properties of an outer surface of the skin, the ability of one or more subcutaneous layers to absorb a specific drug, distance to blood vessels, whether or not the site has been used recently for a similar skin invasive action, etc.

The indicator is configured to generate a visual, audible, and/or tactile indication to a user, and/or to send out a signal with the data about the received ultrasonic waves or the determination of the process based on the received ultrasonic waves. In examples where the indicator can send out a signal, the indicator comprises a communication unit. For example, if the initially selected site is determined suitable, the status indicator will signal the user to proceed and then the skin invasive action can subsequently be performed at that site.

In a preferred example, the indicator has a first state, a second state, and a third state. The first state shows the detector is not activated, the second state indicates that the delivery site is suitable, and the third state signifies that the delivery site is not suitable. After completion of the medicament delivery, the status indicator can return to the first state.

The parameters measured by the detector of the present disclosure can relate to the outer surface of the skin and/or to one or more layers positioned beneath the outer surface of the skin. The algorithm in the detector and part of the processor can confirm whether the area is suitable for injection or not and can also give information about the depth of penetration of the medicament or the outlet orifice, e.g. an injection needle, or both. In some cases, the detector will send a signal to the dose setting and delivery mechanism of the medicament device where such information is used to physically adjust the depth of penetration of the outlet orifice. Such an adjustment can be fully automatic or require some manual manipulation of the dose setting mechanism by the user. For example, the user can be notified that a component of the medicament delivery device needs to be extended or retracted, e.g., the delivery member guard, in order to correct the depth of the penetration of the delivery member.

In general, the correct placement or the unsuitable placement can be information that is collected and processed by the detector which can then be sent to an external device using a communications component in the detector. This is beneficial where the display of the external device is needed to communicate instructions in text and/or graphics to a user.

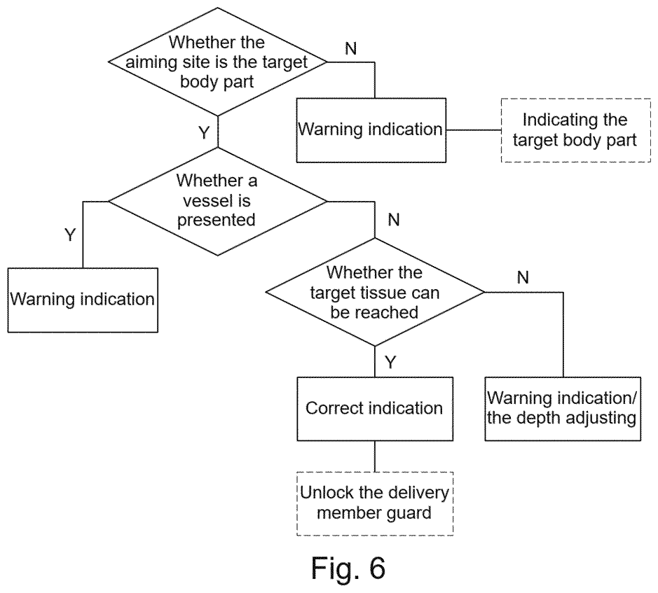

One example of the method of identifying a medicament delivery site is shown in FIG. 6. In this example, the method is performed by the delivery site detector, the processor, the indicator, and optionally a depth adjuster (will be explained in detail later) and an auto-lockout feature (will be explained in detail later). The optional features are shown with dash lines.

It is also possible that the processor is located within another attached supplemental device. For example, the detector may simply transit the received ultrasonic waves signal to a supplemental device, e.g., a smartphone, a computer, or a remote server. The supplemental device processes the signal and sends another instruction to a second supplemental device, e.g., a smartphone or smartwatch, to provide an indication to the user. In this example, the indicator comprises the communication unit and/or the detector comprises a communication unit other than the indicator.

The detector can be powered by a power source, e.g., a battery or a plug or a USB adapter. In a preferred example, the detector comprises a battery so that the medicament delivery device with the detector can be portable. In one example where the detector comprises a battery, the detector can be powered by a small battery that is initially in a disconnected state such that no power is supplied to detector components. The detector can be attached to the medicament delivery device prior to use of the device, for example, during manufacturing or assembly, in a non-energized state where a battery is temporarily electrically isolated from electrical connection with the other components of the detector. In this example, the detector can be active by removing a protective cap of the medicament delivery device that the detector is attached to. In this example, the protective cap comprises a portion of a switch that when activated provides power to the detector. The switch can have an insulating strip having a fixed end attached to the protective cap and a free end positioned between two electrical contacts within a circuit that is operatively connected to the power source and the detector. In one possible embodiment, the detector is powered by a small battery that is initially in a disconnected state such that no power is supplied to detector components. Indeed, the detector can be attached to the medicament delivery device prior to use of the device, for example, during manufacturing or assembly, in a non-energized state where a battery is temporarily electrically isolated from electrical connection with the other components of the detector.

The battery is maintained in an isolated or disconnected state through the use of a mechanically opened normally closed switch or the use of a sheet, ribbon or strip of insulation material that is positioned between two electrical contacts. Holding the normally closed switch open can be achieved through a removable mechanical intervention applied to the switch, such as a switch block that holds the normally closed switch in an open position. Removal of the mechanical intervention will cause the switch to close thus connecting the battery to the circuit. Likewise, the removal of the insulating material will cause electrical contacts to close or otherwise come together to form an electrical connection such that electricity from the battery will flow through and between the contacts, thus closing or energizing the electrical circuit connected to the other electrical components.

The battery that can be used in the activation system of the present disclosure is preferably a button battery, sometimes referred to as a coin or watch battery. Such a battery can be single-use and disposable or can be rechargeable. Preferably, the battery should have a shelf or non-use life of at least several years, most preferably at least 4 years. Further, once activated, the battery should be able to supply power that yields a run time of approximately 30 days and no shorter than 3 weeks. A battery cover can be provided that allows a user access to the battery while the supplemental device is attached to the medicament delivery device housing.

Thus, one of the main ideas of the present disclosure is that the detector is preferably inactive before activation. Depending on the type of technology used, inactive may mean that the circuit is not switched on at all until the battery provides power to the circuitry. Although a conventional coin battery has been described, it is within the scope of the present disclosure that the power or energy source to activate the supplemental device may be derived from less conventional sources, such as a photovoltaic panel or the like.

In one example where the indicator comprises the communication unit or the detector comprises the communication unit, it is preferred that the communication unit is configured to wirelessly transmit data from the detector to an external device when the status indicator is in the second or third state. Information regarding the medicament delivery site can be transmitted to an external device, for example, a mobile device such as a mobile or cell phone, a computer and a remote server such as a cloud for recording, storage and monitoring of the medicament delivery data, such as dosage, time of delivery, date, frequency, type of medicament, etc. A memory unit can also be located in the detector. The detector when part of the supplemental device might further comprise a resetting button which enables a manual reset of the detector. Such a reset feature is beneficial if the supplemental device or part of the supplemental device is to be reused, for example, on another medicament delivery device. The communication unit may also be configured to transmit wirelessly and/or wired data stored in a memory device from the logging device to the computer device external of the medicament delivery device or to a remote location for later analysis.

The data transmission to the external device can occur wirelessly or through a wired connection. The transmission of data between a medicament delivery device and the data collecting device may take place by wireless means, e.g. RF, IR, capacitive or inductive. Transmission of data may take place automatically when the data collecting device and a medicament delivery device are in the proximity of each other, e.g. within a given range. The external or data collecting device may be in the form of one of the following devices: a BGM, a CGM, a drug delivery device, a mechanically controlled drug delivery device, an electronically controlled drug delivery device, a PDA, a mobile phone, a key ring device, a credit card-sized device, a medical hub, router, necklace, smartwatch, or disposable monitoring units. The detector may also comprise an antenna operably connected to the said communication unit and to memory storage elements that contain unique identification data relating to the medicament delivery device.

In one example, the electronics circuit of the detector may comprise Bluetooth technology, which has a few advantages. A Bluetooth transmitter may communicate with a smart device, which may not have to be at such a close range as e.g. NFC technologies. Most smart devices nowadays are arranged with Bluetooth communication circuits, which facilitates the transmission of information from the information transmitted to the smart device. A further advantage with Bluetooth is the possibility that an information transmitter of a medicament delivery device provided to a certain user is bonded to a smart device of a said certain user. There is thus a tight connection between the medicament delivery devices that a user receives and is to use and their personal smart devices. Thus, information from specific medicament delivery devices is only transmitted to specific smart devices.

In one example, the delivery site detector is attached to a delivery member guard of a medicament delivery device. Preferably, the delivery site detector is embedded in the delivery member guard. The delivery member guard comprises a sleeve configured to enclose a delivery member of the medicament delivery device before and after a medicament delivery operation. In a preferred example, the sleeve comprises a proximal wall configured to contact the delivery site. An aperture arranged in the proximal wall of the sleeve is configured to be passed through by the medicament delivery member of the medicament delivery device during use of the medicament delivery device. In this example, the ultrasonic transmitter and the ultrasonic receiver are attached to the proximal wall of the sleeve.

In another example, the delivery site detector has attached a housing of the medicament delivery device. Preferably, the delivery site detector is embedded in the housing.

In another example, the detector is a supplemental device for addition to an existing medicament delivery device. In this example, the detector can be positioned on a housing of an existing medicament delivery device by the manufacturer of the medicament delivery device, by a healthcare provider or by a user of the device. The shape of such a supplemental device preferably matches or conforms to the shape of the housing of the medicament delivery device where it is attached. In the case of a pen-type injection device, the shape of the supplement device could be conical or have a substantially conical-shaped portion adapted to be axially and/or slidably attached to the drug delivery device such that when the supplemental device is mounted it is axially and rotational locked.

The detector or the supplemental device containing the detector may also contain a monitoring device, which could include one or more sensing devices that can be configured to detect a motion of the medicament delivery device. Likewise, a logging device could be included as part of the supplemental device and configured to track user behavior of the medicament delivery device, which user behavior is based on a motion detected by the sensor device. By tracking the motion of the medicament delivery device, tracking of user behavior for study, training or compliance is improved, which facilitates medicament delivery. As used in this disclosure, a sensor device generally refers to any kind of sensor capable of detecting movement. Preferably, the sensor device may comprise one or more of the following sensors: a magnetometer, a gyroscope, and/or an accelerometer, wherein high precision tracking of motion is improved.

The detector or supplemental device containing the detector may further comprise a memory device configured to store data related to the determination of the appropriateness of a chosen medicament delivery site or information generated from a logging device, or related to the communication device. The memory device may comprise non-volatile memory. When the medicament delivery device is a medicament delivery device, the detector may be part of a supplemental device or maybe an integral part of an actual delivery device for injection of a medicament or may be a mock-up demo device for human factors study or for training.

In one example where the delivery site detector is attached to the medicament delivery device instead of as a part of the supplemental device, as shown in FIGS. 1A-2B and FIG. 5. There is shown one possible embodiment 30 of the delivery site detector 40 and medicament delivery device 20, in this example, the detector is built into or integral with the design of the delivery device. The medicament delivery device comprises a housing 24 to accommodate a medicament container. In this particular embodiment, the detector has one or more ultrasound transmitters/receivers 40 and an indicator 41 shown as part of the housing 24 of the delivery device 20. The delivery device 20 has a protective cap 21 protecting the medicament delivery member shown as injection needle 60. The indicator 41 is located on the outside housing 24 and is operatively connected to the detector and the transmitters/receivers 40. The indicator 41 as shown is a “go” (green light) “no go” (red light) indicator where if the user selects a suitable injection site, then the green light is lit, and where an inappropriate injection site has been selected the red light is lit and the delivery device is locked from performing an injection. Preferably, when the detector is activated, neither light is lit, and the delivery device can be locked to prevent activation of any dose setting and/or delivery mechanism.

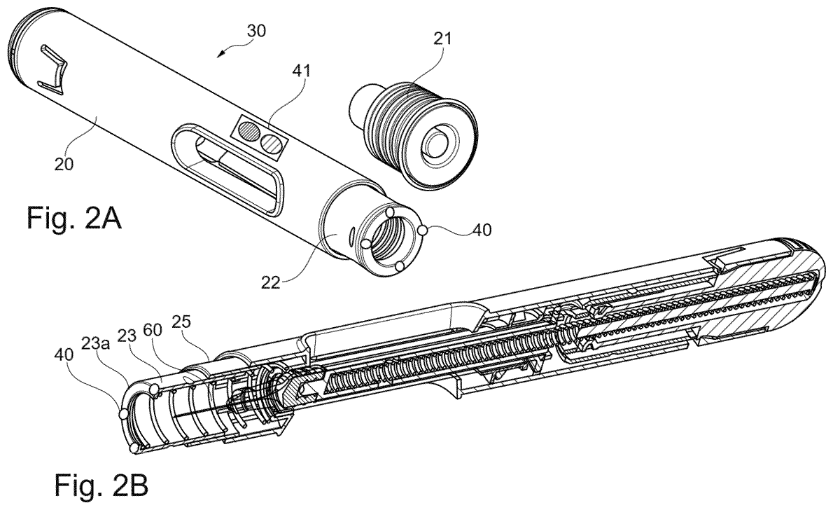

Another possible embodiment of the present disclosure is shown in FIGS. 2A and 2B where basically the same medicament delivery device 20 has the detector setup described above except here the transmitters/receivers 40 are integrated into and made part of a delivery member guard 22. The delivery member guard comprises a sleeve. The sleeve can be formed by a distal portion 25 that can be adjusted and axially moved relative to a proximal end portion 23. The sleeve is slidable relative to the proximal end of the housing 24. The proximal end portion 23 comprises the proximal wall 23a. In the preferred example, the transmitters/receivers 40 are integrated into the proximal wall 23a.

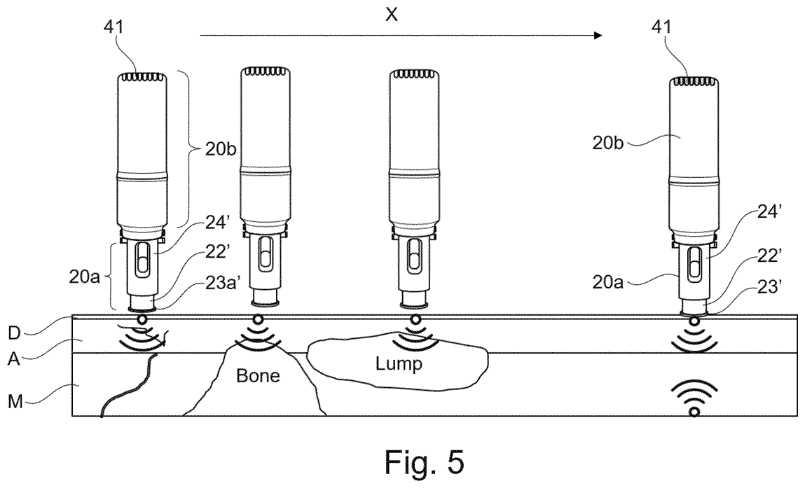

In another example, as shown in FIG. 5, the medicament delivery device comprises a disposable cassette unit 20a and a reusable drive unit 20b releasably attached to the cassette unit. Preferably, the delivery site detector is arranged in the cassette unit 20a. The battery, the indicator 41 and the processor are arranged in the drive unit 20b. In this example, the disposable cassette unit 20a comprises a housing 24′ and a delivery member guard 22′. Similar to the examples above, the delivery site detector 40 can be either integral to the housing 24′ of the cassette unit or the delivery member guard 22′ of the cassette unit 20a. Preferably, the transmitters/receivers 40 are integrated into the proximal wall 23a′ of the delivery member guard 22′ of the cassette unit 20a. In this example, the user can hold and move the medicament delivery device upon the skin (arrow X), the indicator 41 can be inactive, e.g., no light and/or sound and/or vibration is generated; or the indicator 41 can generate an ‘not ready’ signal, e.g., red light, before the predetermined delivery site has been found by the detector 40. When the predetermined delivery site is detected, the indicator 41 generates a ‘ready’ signal, e.g., a green light, sounds, vibration, to indicate the user that the site that is aimed by the medicament delivery device is proper to receive the contained medicament of the medicament delivery device.

In a preferred example, the housing 24′ of the disposable cassette unit 20a extends along a longitudinal axis from a proximal end to a distal end. The delivery member guard 22′ of the cassette unit 20a is telescopically arranged to the proximal end of the housing 24′. In a preferred example, the distal end of the housing 24′ is configured to releasably attached to the reusable drive unit 20b, e.g., by snap-fit, bayonet-fit, or screw threads. In the preferred example, when the proximal wall 23a′ of the delivery member guard 22′ is pressed towards the distal end of the housing 24′, a medicament delivery member of the medicament delivery device can expose, and thus deliver medicament contained within the cassette unit 20a. In one example where the delivery site detector is integral into the proximal wall 23 of the delivery member guard 22′ of the cassette unit, the cassette unit can be arranged with conductive wires. The conductive wires extend from the delivery site detector towards the distal end of the housing 24′. The conductive wires can end at a distal end of the delivery member guard 22′. Preferably, when the cassette unit 20a is attached to the drive unit 20b, the drive unit 20b can power the delivery site detector via the conductive wires; in the meantime, the detection from the detector can also transmit to the processor in the drive unit 20b. The drive unit 20b can connect to the distal end of the delivery member guard via wire or wireless, e.g., RF technology. Preferably, the drive unit 20b is wirelessly connected to the distal end of the delivery member guard when the drive unit 20b connects to the distal end of the housing 24′ of the cassette unit 20a.

In one example, the medicament delivery device comprises a depth adjuster configured to determine the furthest distance that a medicament delivery member can reach.

The depth adjuster can include an axially movable portion of the delivery member guard, where the axial position of the portion of the delivery member guard relative to the housing stop is directly proportional to an end position of the outlet orifice during medicament delivery. The depth adjuster can be a movable hard stop located inside the housing, where the axial positioning of the hard stop is directly proportional to an end position of the outlet orifice during medicament delivery. In some designs of the present disclosure, the depth adjuster is automatically controlled and adjusted based on an adjustment signal received from the detector. A preferred location of the detector is where it is positioned on a proximal end of the device, for example, having the detector located on a terminal proximal end of the guard.

In one example, as shown in FIGS. 2A-2B, the depth of penetration of the injection needle 60 can be adjusted manually by manually adjusting the relative position between the proximal part 23 and the distal part 25 based on input received from the detector 40. The adjustment can be made using a screw connection, a snap-lock connection, or friction fit.

In another example, the depth adjuster is automatically controlled and adjusted based on an adjustment signal received from the detector. In this example, the depth adjuster connects to the processor. The depth adjuster further operably connects to either a medicament container or the delivery member guard 22, 22′. As medicament containers usually are arranged with medicament delivery members, an axial movement of the medicament container thereby adjusting a position of the medicament delivery member. For example, the drive unit 20b can comprise a gripper configured to engage with a medicament container carrier that axially immovably accommodates a medicament container in the cassette unit 20a, when the cassette unit 20a is attached to the drive unit 20b. Thus, the drive unit 20b can axially move the medicament container within the cassette unit 20a, thereby the function of the depth adjuster is achieved.

The processor is configured to control the depth adjuster to move the medicament container when the depth adjuster operably connects to the medicament container to a predetermined position based on the detection of the delivery site detector; or the processor is configured to control the depth adjuster to move the delivery member guard, when the depth adjuster operably connects to the delivery member guard, to a predetermined position based on the detection of the delivery site detector. For example, the depth adjuster can be a motor-driven gear set, the processor can control the depth adjuster to move the medicament container towards the proximal end/distal end of the housing 24, 24′, or control the depth adjuster to move the delivery member guard 22, 22′ towards the proximal end/distal end of the housing 24, 24′, or control the depth adjuster to move the proximal part 23 of the delivery member guard relative to the distal part 25 of the delivery member guard.

In another example, the medicament delivery device provides an auto-lockout feature. The auto-lockout feature is configured to releasably lock the medicament delivery device from being activated or used to perform a dose delivery. Such a lockout could occur when the detector has determined that the chosen delivery site is not suitable. This lockout feature can be performed by locking the delivery member guard to the housing. For example, the medicament delivery device comprises a latch assembly. In one example, the latch assembly can be a solenoid arrangement. In this example, the latch assembly can be a metal latch rod wrapped by coils and a locking groove. It can be designed that either the latch rod will be pulled out from the locking groove or pushed into the locking hole due to the change of electromagnetic field when the coils are connected to an electrical power supply. In this example, one of the latch rod and the locking groove is attached to the delivery member guard, and the other of the latch rod and the locking hole is attached to the housing. The processor is configured to switch on/off the electrical power supply of the coils based on the detection of the delivery site detector.

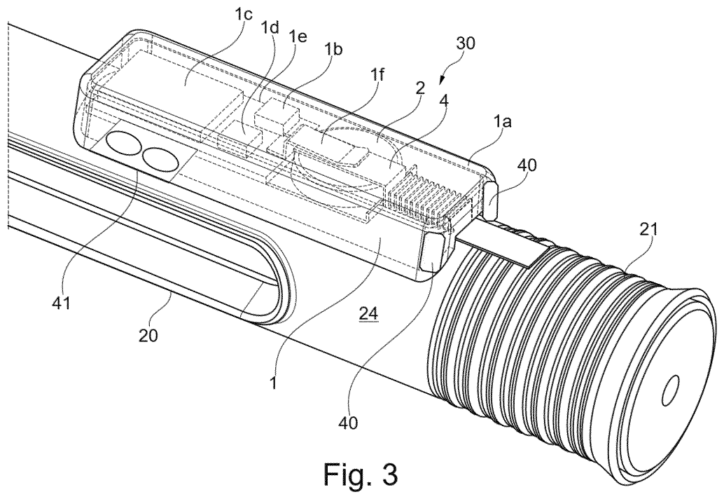

Yet another possible embodiment 30 of the present disclosure is shown in FIG. 3, where the detector is contained in a supplemental device 1. The transmitters/receivers 40 are located on a proximal end face of the supplemental device 1 such that they will be directed toward a selected injection site. The supplemental device 1 can be attached to the outer housing 24 of the injection device 20 having a protective cap 21 and containing a medicament container 54 (see FIG. 1B) having a fixed needle 60 that is covered by needle shield 62. A needle shield remover is attached to the protective cap 21, where the removal of the cap 21 from housing 24 necessarily causes removal of the needle shield from needle 60. Attached to the cap 21 is part of a blocking element made up of tether 4 which can be comprised of an insulating material such that a portion of the tether is located between the biasing element If and battery 2. A portion of the tether can be stored inside the device 1 in a folded or accordion configuration or any such configuration that will hold the excess of the tether when the cap is attached, such as a wrapped configuration. The movement of cap 21 away from housing 24 will cause the blocking element to be removed from the device 1, thus closing the electrical circuit and allowing the battery to energize the detector electrical components. Also included in device 1 are a number of electrical components such as a communication device 1b, a recorder 1d having data storage, and a processor or control unit 1c. All of these components are in electrical communication through circuit board le and enclosed by cover 1a.

Alternatively, when the delivery site detector is the supplemental device, the delivery site detector can be attached to the medicament delivery device that comprises the disposable cassette unit and the reusable drive unit as mentioned above. In this example, the delivery site detector can be attached to the housing of the disposable cassette unit. In one example, the delivery site detector can comprise a battery. Alternatively, the delivery site detector can connect to the reusable drive unit via wire.

The medicament delivery device will typically include as part of the dose setting and delivery mechanism a component for performing the skin invasive action, i.e., the delivery member, for example, may be in the form of an injection needle for use in those cases where the skin invasive action comprises injecting a drug into the part of the skin. Alternatively, or additionally, the skin invasive action may involve the use of an infusion apparatus, e.g. for infusing a drug or a fluid into the mammal. Likewise, the skin invasive action may be necessary for the collection of a blood sample, a tissue sample and/or a sample of another kind of body fluid, e.g. interstitial fluid. This may include a needle or biopsy equipment for obtaining a biopsy of one or more subcutaneous layers. The skin invasive action may also involve a needle containing a sensor device for measuring one or more components of a body fluid, e.g. interstitial fluid, like a continuous plasma glucose sensor. Additionally, the skin invasive action may be a result of a jet injector adapted to inject a drug or a fluid into a subject by means of jet injection, i.e. a ‘needleless’ injection where the drug or fluid is injected by means of relatively high pressure. Such devices may advantageously be used for inoculations.

The inventive concept has mainly been described above with reference to a few examples. However, as is readily appreciated by a person skilled in the art, other embodiments than the ones disclosed above are equally possible within the scope of the inventive concept, as defined by the appended claims.

Some aspects of the invention are described in the clauses below.
1. A delivery site detector configured to be attached to a medicament delivery device, the delivery site detector comprising:
   an ultrasonic transmitter and an ultrasonic receiver;
   wherein the ultrasonic transmitter is configured to transmit an ultrasonic wave to a delivery site; wherein the ultrasonic receiver is configured to receive an ultrasonic wave reflected by the delivery site; and
   wherein the ultrasonic transmitter comprises a frequency generator configured to generate and transmit ultrasonic waves with at least two different ultrasound frequencies.
2. The delivery site detector according to clause 1, wherein the frequency generator comprises a plurality of stacked dielectric or piezoelectric layers.
3. The delivery site detector according to clause 2, wherein at least two of the plurality of stacked dielectric or piezoelectric layers have different thicknesses.
4. The delivery site detector according to clause 2, wherein all layers of the plurality of stacked dielectric or piezoelectric layers have the same thickness.
5. The delivery site detector according to any one of clauses 2-4, wherein each of the plurality of stacked dielectric or piezoelectric layers is bonded to its adjacent dielectric or piezoelectric layers with conductive material coating on each of the plurality of stacked dielectric or piezoelectric layers.
6. The delivery site detector according to any of the preceding clauses, comprising:
   an indicator operably connected to the delivery site detector;
   a processor connected to the indicator and the delivery site detector;
   a battery connected to the delivery site detector, the indicator, and the processor;
   wherein the processor is configured to control the indicator to provide at least a visual, audible, and/or tactile indication, and/or to send out a signal containing data based on a detection of the delivery site detector.
7. A delivery member guard of a medicament delivery device, the delivery member guard comprising the delivery site detector according to any one of clauses 1-6.
8. The delivery member guard according to clause 7, wherein the delivery member guard comprises a sleeve; wherein the sleeve comprises a proximal wall configured to contact the delivery site; wherein an aperture is arranged in the proximal wall of the sleeve and is configured to be passed through by a medicament delivery member of the medicament delivery device during use of the medicament delivery device; and wherein the ultrasonic transmitter and the ultrasonic receiver are attached to the proximal wall of the sleeve.
9. A housing of a medicament delivery device, wherein the housing is configured to accommodate a medicament container of the medicament delivery device, the housing comprising the delivery site detector according to any one of clauses 1-6.
10. A medicament delivery device comprising either the delivery member guard according to any of clauses 7-8, or the housing according to clause 9.
11. The medicament delivery device according to clause 10, when comprising the delivery site detector according to any of clauses 1-6, the medicament delivery device comprising:
   an indicator operably connected to the delivery site detector;
   a processor connected to the indicator and the delivery site detector;
   a battery connected to the delivery site detector, the indicator, and the processor;
   wherein the processor is configured to control the indicator to provide at least a visual, audible, and/or tactile indication based on a detection of the delivery site detector, and/or to send out a signal containing data based on a detection of the delivery site detector.
12. The medicament delivery device according to clause 11, comprising a disposable cassette unit and a reusable drive unit releasably attached to the cassette unit; wherein the delivery site detector is arranged in the cassette unit; and wherein the battery, the indicator and the processor are arranged in the drive unit.
13. The medicament delivery device according to clause 11 or 12, comprising a removable protective cap for sealing a medicament delivery member; wherein the protective cap comprises a portion of a switch that when activated provides power to the delivery site detector.
14. The medicament delivery device according to clause 13, wherein the switch comprises an insulating strip having a fixed end attached to the protective cap and a free end positioned between two electrical contacts within a circuit that is operatively connected to the battery and the detector.
15. The medicament delivery device according to any of clauses 10-14, comprising a depth adjuster configured to determine the furthest distance that a medicament delivery member can reach.
16. The medicament delivery device according to clause 15, wherein the depth adjuster is automatically controlled and adjusted based on an adjustment signal received from the detector.
17. The medicament delivery device according to clause 16 when dependent on any of clauses 10-14, wherein the depth adjuster connects to the processor; wherein the depth adjuster operably connects to either a medicament container or a delivery member guard; and wherein the processor is configured to control the depth adjuster to move the medicament container when the depth adjuster operably connects to the medicament container to a predetermined position based on the detection of the delivery site detector; or the processor is configured to control the depth adjuster to move the delivery member guard, when the depth adjuster operably connects to the delivery member guard, to a predetermined position based on the detection of the delivery site detector.
18. A method of identifying a medicament delivery site, the method comprising the following steps in the following order:
   providing a medicament delivery device with the delivery site detector according to any of clauses 1-6;
   transmitting ultrasonic waves from the delivery site detector with at least two different ultrasonic frequencies towards a selected delivery site;
   receiving the ultrasonic waves from the selected delivery site;
   determining, based on the received ultrasonic waves, whether the selected delivery site is suitable for delivery of the contained medicament of the medicament delivery device.
19. The method according to clause 18, the method further comprising a step of: determining whether the penetration depth of a delivery member of the medicament delivery device needs to be adjusted if the selected delivery site is suitable for delivery of the contained medicament of the medicament delivery device.
20. The method according to clause 18 or 19, the method further comprising a step of: providing at least one audible, visual, and/or tactile indication, and/or sending out a signal containing data based on the determination of whether the selected delivery site is suitable for delivery of the contained medicament of the medicament delivery device.
21. The method according to any of clause 19-21, when the delivery site detector is the delivery site detector according to any of clauses 2-7, wherein the step of transmitting ultrasonic waves from the delivery site detector further comprises the step: selectively providing a voltage across varying numbers of dielectric or piezoelectric layers to generate ultrasonic waves with at least two different ultrasonic frequencies.

1-15. (canceled)

16. A delivery site detector configured to be attached to a medicament delivery device, the delivery site detector comprising:
an ultrasonic transmitter; and
an ultrasonic receiver,
wherein the ultrasonic transmitter is configured to transmit an ultrasonic wave to a delivery site,
wherein the ultrasonic receiver is configured to receive an ultrasonic wave reflected by the delivery site, and
wherein the ultrasonic transmitter comprises a frequency generator configured to generate and transmit ultrasonic waves with at least two different ultrasound frequencies.

17. The delivery site detector according to claim 16, wherein the frequency generator comprises a plurality of stacked dielectric or piezoelectric layers.

18. The delivery site detector according to claim 17, wherein at least two of the plurality of stacked dielectric or piezoelectric layers have different thicknesses.

19. The delivery site detector according to claim 17, wherein all layers of the plurality of stacked dielectric or piezoelectric layers have the same thickness.

20. The delivery site detector according to claim 17, wherein each of the plurality of stacked dielectric or piezoelectric layers is bonded to its adjacent dielectric or piezoelectric layers with conductive material coating on each of the plurality of stacked dielectric or piezoelectric layers.

21. The delivery site detector according to claim 16, comprising:
an indicator operably connected to the delivery site detector;
a processor connected to the indicator and the delivery site detector; and
a battery connected to the delivery site detector, the indicator, and the processor,
wherein the processor is configured to control the indicator to provide at least a visual, audible, and/or tactile indication, and/or to send out a signal containing data based on a detection of the delivery site detector.

22. The delivery site detector according to claim 16, wherein the ultrasonic transmitter and the ultrasonic receiver are formed in one transducer.

23. A delivery member guard of a medicament delivery device, the delivery member guard comprising the delivery site detector according to claim 16.

24. The delivery member guard according to claim 23, wherein the delivery member guard comprises a sleeve, wherein the sleeve comprises a proximal wall configured to contact the delivery site, wherein an aperture is arranged in the proximal wall of the sleeve and is configured to be passed through by a medicament delivery member of the medicament delivery device during use of the medicament delivery device, and wherein the ultrasonic transmitter and the ultrasonic receiver are attached to the proximal wall of the sleeve.

25. A housing of a medicament delivery device, wherein the housing is configured to accommodate a medicament container of the medicament delivery device, the housing comprising the delivery site detector according to claim 16.

26. A medicament delivery device comprising either the delivery member guard according to claim 23, or the housing according to claim 25.

27. The medicament delivery device according to claim 26, when comprising the delivery site detector according to claim 16, the medicament delivery device comprising:
an indicator operably connected to the delivery site detector;
a processor connected to the indicator and the delivery site detector; and
a battery connected to the delivery site detector, the indicator, and the processor,
wherein the processor is configured to control the indicator to provide at least a visual, audible, and/or tactile indication based on a detection of the delivery site detector, and/or to send out a signal containing data based on a detection of the delivery site detector.

28. The medicament delivery device according to claim 27, comprising a disposable cassette unit and a reusable drive unit releasably attached to the cassette unit, wherein the delivery site detector is arranged in the cassette unit, and wherein the battery, the indicator and the processor are arranged in the drive unit.

29. The medicament delivery device according to claim 26, comprising a depth adjuster configured to determine the furthest distance that a medicament delivery member can reach.

30. The medicament delivery device according to claim 29, wherein the depth adjuster is automatically controlled and adjusted based on an adjustment signal received from the detector.
